(19) United States
(12) Patent Application Publication
(10) Pub. No.: US 2026/0055365 A1
(43) Pub. Date: Feb. 26, 2026

(54) METHOD FOR SELECTIVE DIFFERENTIATION FROM PLURIPOTENT STEM CELLS TO HINDBRAIN TISSUE

(71) Applicant: NEXT&BIO INC., Seoul (KR)

(72) Inventors: Tae Hwan Kwak, Gyeonggi-do (KR); Ji Hun Yang, Seoul (KR)

(73) Assignee: NEXT&BIO INC., Seoul (KR)

(21) Appl. No.: 19/102,632

(22) PCT Filed: Aug. 9, 2023
(86) PCT No.: PCT/KR2023/011785

§ 371 Date: Feb. 10, 2025

(30) Foreign Application Priority Data
Aug. 10, 2022 (KR) 10-2022-0099739

(51) Int. Cl. C12N 5/0793

(52) U.S. Cl. CPC C12N 5/0619; C12N 2501/415; C12N 2501/727; C12N 2506/45

(57) ABSTRACT

The present disclosure relates to a method for selective differentiation from pluripotent stem cells to hindbrain tissue, the method comprising a step of selective differentiation into hindbrain tissue, in which embryoid bodies formed from pluripotent stem cells are cultured in the presence of a hindbrain tissue induction medium containing endogenous WNT secretion inhibitors and WNT signaling agonists.

### TECHNICAL FIELD

The present disclosure relates to a method for selective differentiation from pluripotent stem cells to hindbrain tissue.

### BACKGROUND ART

An organoid, also called ‘organ analogue’ or ‘organ-like body’, is an organ-specific cell aggregate prepared by reaggregating and recombining cells isolated from stem cells or organ-origin cells using a 3D culture method. The organoid contains specific cells of the organ for modeling, reproduces specific functions of the organ, and may be spatially organized in a form similar to an actual organ.

In particular, organoids may be prepared to mimic most human organs using pluripotent stem cells that may differentiate into all cells of the human body, and among these, a brain organoid has received a lot of attention as an in vitro culture model for mimicking various intractable central nervous system diseases to elucidate the pathogenesis and develop treatments.

For example, Lancaster et al. prepared brain organoids using human induced pluripotent stem cells (Non-Patent Document 1). However, the brain organoids prepared in this way have a problem that various organs, such as the cerebrum, midbrain, and retina, are mixed in a single organoid, and their sizes are also different.

Most intractable brain diseases are diseases that occur specifically in a specific brain region, and organoids that have the characteristics of a brain region other than a target disease site or have mixed characteristics of various regions may not accurately mimic brain diseases. Therefore, in order to utilize brain organoids for research on the pathogenesis of intractable brain diseases through modeling, research on developing treatments, etc., it is required to develop a region-specific brain organoid fabrication technology capable of mimicking a specific brain region with high purity.

### PRIOR ARTS

### Non-Patent Document

(Non-Patent Document 1) Madeline A. Lancaster, Juergen A. Knoblich, Generation of Cerebral Organoids from Human Plurpotent Stem Cells, Nat Protoc. 2014 October; 9 (10): 2329-2340.

### DISCLOSURE

### Technical Problem

An object of the present disclosure is to provide a method for selective differentiation from pluripotent stem cells to hindbrain tissue, thereby providing a basis for preparing region-specific brain organoids.

However, technical objects of the present disclosure are not limited to the aforementioned purpose and other objects which are not mentioned may be clearly understood to those skilled in the art from the following description.

### Technical Solution

An aspect of the present disclosure provides a method for selective differentiation from pluripotent stem cells to hindbrain tissue, the method including: a step of selective differentiation into hindbrain tissue, in which embryoid bodies formed from pluripotent stem cells are cultured in the presence of a hindbrain tissue induction medium containing an endogenous WNT secretion inhibitor and a WNT signaling agonist.

### Advantageous Effects

According to the present disclosure, by the method for selective differentiation from the pluripotent stem cell to hindbrain tissue, it is possible to uniformly differentiate embryoid bodies formed from pluripotent stem cells into a hindbrain region-specific cell aggregate. Accordingly, it is possible to provide a basis for preparing a specific brain organoid by inducing selective differentiation into the pons and cerebellum.

### DESCRIPTION OF DRAWINGS

FIG. 1 is a schematic diagram showing a differentiation mechanism of a method for selective differentiation from pluripotent stem cells into hindbrain tissue according to one embodiment of the present disclosure.

FIG. 2 shows results of expression of forebrain region-specific genes according to a concentration of a WNT signaling agonist in a hindbrain tissue induction medium of Example.

FIG. 3 shows results of expression of hindbrain region-specific genes according to a concentration of a WNT signaling agonist in a hindbrain tissue induction medium of Example.

FIG. 4 shows results of expression of spinal cord region-specific genes according to a concentration of a WNT signaling agonist in a hindbrain tissue induction medium of Example.

### BEST MODE

Hereinafter, the present disclosure will be described in detail.

The present disclosure may have various modifications and various embodiments and specific embodiments will be illustrated in the drawings and described in detail in the detailed description.

However, the present disclosure is not limited to specific embodiments, and it should be understood that the present disclosure covers all the modifications, equivalents and replacements included within the idea and technical scope of the present disclosure. In describing the present disclosure, when it is determined that a detailed description of related known arts may obscure the gist of the present disclosure, the detailed description will be omitted.

Terms used herein are used only to describe specific embodiments, and are not intended to limit the present disclosure. A singular expression includes a plural expression unless otherwise defined differently in a context.

In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations of these, in advance.

An embodiment of the present disclosure provides a method for selective differentiation from pluripotent stem cells to hindbrain tissue, the method including: a step of selective differentiation into hindbrain tissue, in which embryoid bodies formed from pluripotent stem cells are cultured in the presence of a hindbrain tissue induction medium containing an endogenous WNT secretion inhibitor and a WNT signaling agonist.

According to one embodiment of the present disclosure, through the step of selective differentiation into the hindbrain tissue, the embryoid bodies formed from the pluripotent stem cells may differentiate into a cell aggregate that mimics hindbrain characteristics with high purity. Specifically, the cell aggregate mimicking the hindbrain characteristics may be an organoid mimicking the hindbrain characteristics with high purity. The cell aggregate or organoid mimicking the hindbrain characteristics with high purity may mean a state in which the mixing of tissues exhibiting characteristics of other brain regions such as the cerebrum, striatum, and midbrain is minimized.

According to one embodiment of the present disclosure, the step of selective differentiation into the hindbrain tissue may uniformly induce patterning into hindbrain tissue throughout the embryoid bodies, while inducing differentiation of the embryoid bodies into the neuroectoderm.

According to one embodiment of the present disclosure, the endogenous WNT secretion inhibitor may be a material that inhibits the secretion of a WNT signaling material naturally expressed in the embryoid bodies. The endogenous WNT secretion inhibitor may control the heterogeneous expression of an uncontrollable WNT signaling pathway by inhibiting the activation of the WNT signaling pathway by means of a WNT protein spontaneously expressed within the embryoid bodies. Therefore, the embryoid bodies may be selectively and homogeneously differentiated into the hindbrain by combining and applying the endogenous WNT secretion inhibitor and a WNT signaling agonist to be described below. The endogenous WNT secretion inhibitor may include a material capable of performing a mechanism as a Porcupine O-Acyltransferase (PORCN) inhibitor. Specifically, the endogenous WNT secretion inhibitor may include at least one selected from the group consisting of IWP-2, LGK-974, ETC-159, GNF6231, WNT-C59, and WNT974. More specifically, the endogenous WNT secretion inhibitor may include IWP-2. More specifically, the endogenous WNT secretion inhibitor may effectively neutralize the activity of the WNT signaling pathway by inhibiting the secretion of naturally expressed WNT signaling materials, and simultaneously may not affect the activity thereof even if the WNT signaling agonist is treated. That is, the endogenous WNT secretion inhibitor may effectively control the heterogeneous formation of the WNT signaling pathway by inhibiting a process of transforming the endogenous WNT protein spontaneously expressed in the embryoid bodies into a form secreted out of cells from the endoplasmic reticulum. In contrast, materials such as IWR-1-endo may have a problem of reducing an effect of the WNT signaling agonist to be described below, by inhibiting signaling in a lower cascade of the WNT signaling pathway.

According to one embodiment of the present disclosure, the hindbrain tissue induction medium may include an endogenous WNT secretion inhibitor in a concentration range that exhibits an effect equivalent to the effect exhibited by IWP-2 in a concentration range of 0.1 μM or more and 2 μM or less. Specifically, the hindbrain tissue induction medium may include an endogenous WNT secretion inhibitor in a concentration range that exhibits an effect equivalent to that exhibited by IWP-2 in a concentration range of 0.5 μM or more and 2 μM or less, 0.5 μM or more and 1.5 μM or less, or 0.8 μM or more and 1.2 μM or less. In addition, according to one embodiment of the present disclosure, the hindbrain tissue induction medium may include IWP-2 in a concentration range of the endogenous WNT secretion inhibitor. When the concentration of the endogenous WNT secretion inhibitor is within the above range, the formation of the WNT signaling pathway spontaneously expressed in the embryoid bodies may be effectively inhibited. Specifically, when the concentration of the endogenous WNT secretion inhibitor is less than the above range, an effect of controlling the heterogeneous expression of the uncontrollable WNT signaling pathway may be minimal, resulting in the heterogeneous formation of the WNT signaling pathway. In addition, when the concentration of the endogenous WNT secretion inhibitor is more than the above range, excessive inhibition of the WNT signaling pathway may result in decreased cell division and/or decreased cell viability.

According to one embodiment of the present disclosure, the WNT signaling agonist may uniformly impart hindbrain region specificity to the entire embryoid bodies. That is, as described above, while the endogenous WNT secretion inhibitor inhibits the generation of the uncontrollable WNT signaling material in the embryoid bodies, the WNT signaling agonist may uniformly control the WNT signal intensities within the entire embryoid bodies to have uniform hindbrain specificity throughout the embryoid bodies.

According to one embodiment of the present disclosure, the WNT signaling agonist may include at least one of a protein-based WNT signaling agonist and a compound-based WNT signaling agonist. Specifically, the protein-based WNT signaling agonist may include at least one selected from the group consisting of Wnt Family Member 1 (WNT-1), Wnt Family Member 3A (WNT-3a), and R-spondin-1 (RSPO1). In addition, the compound-based WNT signaling agonist may include a material that performs the mechanism of a GSK-3 beta inhibitor, and specifically, may include at least one selected from the group consisting of CHIR99021, CP21R7, CHIR98014, LY2090314, kenpaullone, AR-AO144-18, TDZD-8, SB216763, BIO, TWS-119, and SB415286. Specifically, the WNT signaling agonist may be CHIR99021.

According to one embodiment of the present disclosure, the selective differentiation into the hindbrain region may be induced by optimizing the concentration of the WNT signaling agonist in the hindbrain tissue induction medium. Specifically, the concentration of the WNT signaling agonist for inducing selective differentiation of the embryoid bodies into the hindbrain region may be as follows.

According to one embodiment of the present disclosure, the hindbrain tissue induction medium may include a WNT signaling agonist in a concentration range that exhibits an effect equivalent to the effect exhibited by CHIR99021 in a concentration range of more than 1 μM and less than 5 μM. Specifically, the hindbrain tissue induction medium may include a WNT signaling agonist in a concentration range that exhibits an effect equivalent to the effect exhibited by CHIR99021 in a concentration range of 1.5 μM or more and 4.5 μM or less, 1.5 μM or more and 4 μM or less, 1.5 μM or more and 3 μM or less, or 1.5 μM or more and 2.5 μM or less. More specifically, the hindbrain tissue induction medium may include the WNT signaling agonist at a concentration that specifically expresses at least one gene of LMX1A and LMX1B. In addition, according to one embodiment of the present disclosure, the hindbrain tissue induction medium may include CHIR99021 in a concentration range of the WNT signaling agonist. When the concentration of the WNT signaling agonist is within the above range, the expression of hindbrain region-specific genes (e.g., LMX1A and/or LMX1B genes) is effectively greatly increased, so that the embryoid bodies may be uniformly differentiated into a cell population having hindbrain specificity. When the concentration of the WNT signaling agonist is less than the above range, the expression of forebrain region-specific genes (e.g., FOXG1, LHX2, and/or DLX2 genes) rather than the hindbrain region may be increased. In addition, when the concentration of the WNT signaling agonist is more than the above range, the expression of spinal cord region-specific genes (e.g., HOXB4, HOXC9 and/or HOXA 1 genes) rather than the hindbrain region may be increased.

According to one embodiment of the present disclosure, in the step of selective differentiation into the hindbrain tissue, at least one gene of LMX1A and LMX1B may be expressed.

According to one embodiment of the present disclosure, the pluripotent stem cells may be human-derived embryonic stem cells or human-derived induced pluripotent stem (iPS) cells. The pluripotent stem cells refer to stem cells that may differentiate into three germ layers of endoderm, mesoderm, and ectoderm (pluripotency), and may include induced pluripotent stem cells (iPS) and adult stem cells, which have the capabilities, as well as the embryonic stem cells.

According to one embodiment of the present disclosure, the step of selective differentiation into the hindbrain tissue may include: a1) a step of forming embryoid bodies from pluripotent stem cells in an embryoid body formation medium; and a2) a step of adding a hindbrain tissue induction medium containing an endogenous WNT secretion inhibitor and a WNT signaling agonist to the embryoid body formation medium.

According to one embodiment of the present disclosure, the embryoid body formation medium may be a liquid medium, and may be applied as long as 3D culture of the pluripotent stem cells is enabled. Furthermore, the embryoid body formation medium may include various known additives for culturing the pluripotent stem cells, depending on the purpose.

According to one embodiment of the present disclosure, the hindbrain tissue induction medium may include a known culture solution and additives capable of culturing the embryoid bodies, and may further include the endogenous WNT secretion inhibitor and the WNT signaling agonist.

According to one embodiment of the present disclosure, the added amount of the hindbrain tissue induction medium may be 0.1 to 2 times greater than a volume of the embryoid body formation medium. More specifically, the added amount of the hindbrain tissue induction medium may be equivalent to the volume of the embryoid body formation medium.

According to one embodiment of the present disclosure, the step of selective differentiation into the hindbrain tissue may further include a3) a step of removing a portion of the entire medium and adding the hindbrain tissue induction medium at one time point of 1 to 3 days. Specifically, the step a3) may be removing the medium equivalent to the amount of hindbrain tissue induction medium added in the step a2) approximately every two days, and adding an equivalent amount of hindbrain tissue induction medium. Through this, nutrients may be supplied to the embryoid bodies, and the endogenous WNT secretion inhibitor and the WNT signaling agonist may effectively act.

According to one embodiment of the present disclosure, the step of selective differentiation into the hindbrain tissue may be performed for a period of 4 to 10 days. Specifically, the step of selective differentiation into the hindbrain tissue may be performed for a period of 5 to 8 days, a period of 5 to 7 days, or a period of about 6 days.

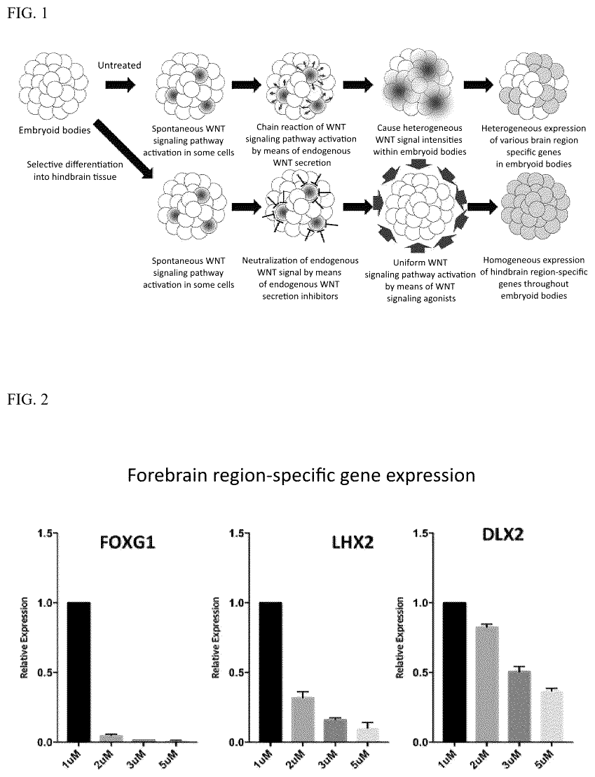

FIG. 1 is a schematic diagram showing a differentiation mechanism of a method for selective differentiation from pluripotent stem cells into hindbrain tissue according to one embodiment of the present disclosure. In FIG. 1, in the case of embryoid bodies that have not been separately treated, a spontaneous and heterogeneous WNT signaling pathway in some cells was activated in the embryoid bodies formed from pluripotent stem cells, so that genes in various brain regions were expressed within one embryoid body. In contrast, when the method for selective differentiation into hindbrain tissue according to the present disclosure is applied to the embryoid bodies formed from the pluripotent stem cells, the spontaneous activation of the WNT signaling pathway in the embryoid bodies may be inhibited, and genes having desired hindbrain region specificity may be uniformly expressed throughout the embryoid bodies.

The present disclosure may have various modifications and various embodiments, and specific embodiments will be hereinafter illustrated in the drawings and described in detail in the detailed description. However, the present disclosure is not limited to specific embodiments, and it should be understood that the present disclosure covers all the modifications, equivalents and replacements within the idea and technical scope of the present disclosure. In describing the present disclosure, when it is determined that a detailed description of related known arts may obscure the gist of the present disclosure, the detailed description will be omitted.

### Examples

### [Formation of Embryoid Bodies]

Pluripotent stem cells were cultured in an embryoid body formation medium as shown in Table 1 below.

TABLE 1

| Ingredient | Product name | Concentration | | Volume | |
|---|---|---|---|---|---|
| DMEM/F12 | Corning, 10-090-CV | 1X | | 37 | ml |
| KSR | ThermoFisher, 10828-028 | 1X | | 10 | ml |
| Penicillin- | ThermoFisher, 15140-122 | 100X | | 0.5 | ml |
| Streptomycin | | | | | |
| Glutamax | ThermoFisher, 35050-061 | 100X | | 0.5 | ml |
| NEAA | ThermoFisher, 11140-050 | 100X | | 0.5 | ml |
| FBS | ThermoFisher, 16000-044 | 1X | | 1.5 | ml |
| Heparin | Sigma-Aldrich, H3149 | 1 | mg/ml | 50 | ul |
| Beta- | ThermoFisher, 21985-023 | 1,000X | | 50 | ul |
| mercaptoethanol | | | | | |
| bFGF | Peprotech, 100-18B | 4 | μg/ml | 50 | ul |
| Y 27632 | Peprotech, 1293823 | 50 | mM | 50 | ul |

| | |
|---|---|
| Total | 50 ml |

When the density of pluripotent stem cells reached approximately 80%, residues on the cell surface were washed using D-PBS and separated into each cell. Furthermore, the cells were diluted to contain approximately 100,000 cells per 10 ml of the embryoid body formation medium, and approximately 100 μl of the medium containing the cells was injected into each well of an Ultra-Low-attachment U bottom 96 well plate (Corning, 7007). Then, the cells were added in an incubator under a CO₂ atmosphere at 37° C. and cultured for about 24 hours to form embryoid bodies.

[Selective Differentiation into Hindbrain Tissue]

After the embryoid bodies were formed as described above, after about one day, it was confirmed whether one spherical embryoid body with a clean surface was formed well in each well. A hindbrain tissue induction medium containing IWP-2 as an endogenous WNT secretion inhibitor at a concentration of 1 μM and CHIR99021 as a WNT signaling agonist at concentrations of 1 μM, 2 μM, 3 μM, and 5 μM was prepared, and then about 100 μl of the hindbrain tissue induction medium was added to each well containing the embryoid body.

About 100 μl of the existing medium was removed once every two days, about 100 μl of the hindbrain tissue induction medium was added, and the embryoid bodies were cultured for 7 days to confirm whether selective differentiation of the embryoid bodies into hindbrain tissue was performed well.

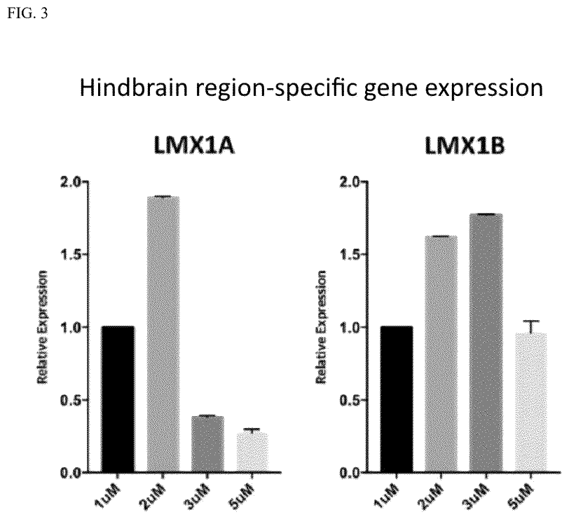

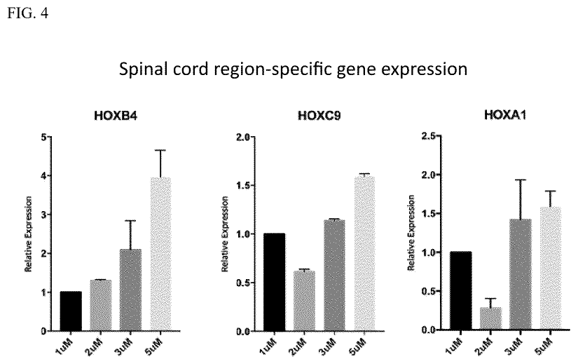

FIG. 2 shows results of expression of forebrain region-specific genes according to a concentration of a WNT signaling agonist in a hindbrain tissue induction medium of Example. FIG. 3 shows results of expression of hindbrain region-specific genes according to a concentration of a WNT signaling agonist in a hindbrain tissue induction medium of Example. Further, FIG. 4 shows results of expression of spinal cord region-specific genes according to a concentration of a WNT signaling agonist in a hindbrain tissue induction medium of Example.

According to the results of FIGS. 2 to 4, it was confirmed that the expression of hindbrain region-specific genes was strongly induced in the hindbrain tissue induction medium containing CHIR99021 in a concentration range of more than 1 μM and less than 5 μM. Furthermore, it was confirmed that the expression intensities of hindbrain region-specific genes were much stronger than those of other brain region-specific genes in the hindbrain tissue induction medium containing CHIR99021 at a concentration range of 2 μM to 3 μM. In contrast, in the case of the hindbrain tissue induction medium containing CHIR99021 at a level of approximately 1 μM, it was confirmed that the expression of forebrain region-specific genes was stronger than that of other brain regions, and in the case of the hindbrain tissue induction medium containing CHIR99021 at a level of approximately 5 μM, it was confirmed that the expression of spinal cord region-specific genes was stronger than that of other brain regions.

1. A method for selective differentiation from pluripotent stem cells to hindbrain tissue, the method comprising:
a step of selective differentiation into hindbrain tissue, in which embryoid bodies formed from pluripotent stem cells are cultured in the presence of a hindbrain tissue induction medium containing an endogenous WNT secretion inhibitor and a WNT signaling agonist.

2. The method of claim 1, wherein the step of selective differentiation into the hindbrain tissue comprises
a1) a step of forming embryoid bodies from pluripotent stem cells in an embryoid body formation medium; and
a2) a step of adding a hindbrain tissue induction medium containing an endogenous WNT secretion inhibitor and a WNT signaling agonist to the embryoid body formation medium.

3. The method of claim 1, wherein the endogenous WNT secretion inhibitor includes at least one selected from the group consisting of IWP-2, LGK-974, ETC-159, GNF6231, WNT-C59, and WNT974.

4. The method of claim 1, wherein the hindbrain tissue induction medium includes an endogenous WNT secretion inhibitor in a concentration range that exhibits an effect equivalent to the effect exhibited by IWP-2 in a concentration range of 0.1 μM or more and 2 μM or less.

5. The method of claim 1, wherein the WNT signaling agonist includes at least one of a protein-based WNT signaling agonist and a compound-based WNT signaling agonist.

6. The method of claim 5, wherein the protein-based WNT signaling agonist includes at least one selected from the group consisting of Wnt Family Member 1 (WNT-1), Wnt Family Member 3A (WNT-3a), and R-spondin-1 (RSPO1).

7. The method of claim 5, wherein the compound-based WNT signaling agonist includes at least one selected from the group consisting of CHIR99021, CP21R7, CHIR98014, LY2090314, kenpaullone, AR-AO144-18, TDZD-8, SB216763, BIO, TWS-119 and SB415286.

8. The method of claim 1, wherein the hindbrain tissue induction medium includes a WNT signaling agonist in a concentration range that exhibits an effect equivalent to the effect exhibited by CHIR99021 in a concentration range of more than 1 μM and less than 5 μM.

9. The method of claim 1, wherein in the step of selective differentiation into the hindbrain tissue, at least one gene of LMX1A and LMX1B is expressed.

10. The method of claim 1, wherein the pluripotent stem cells are human-derived embryonic stem cells or human-derived induced pluripotent stem (iPS) cells.
